(19) United States
(12) Patent Application Publication
(10) Pub. No.: US 2021/0353155 A1
(43) Pub. Date: Nov. 18, 2021

(54) BODY TEMPERATURE DETECTION SAFETY KIOSK

(71) Applicants: Lawrence B. Caplin, Pipersville, PA (US); Zachary D. Caplin, Pipersville, PA (US); Logan C. Caplin, Pipersville, PA (US)

(72) Inventors: Lawrence B. Caplin, Pipersville, PA (US); Zachary D. Caplin, Pipersville, PA (US); Logan C. Caplin, Pipersville, PA (US)

(21) Appl. No.: 17/323,186

(22) Filed: May 18, 2021

Related U.S. Application Data
(60) Provisional application No. 63/026,235, filed on May 18, 2020.

(51) Int. Cl. A61B 5/01; G01J 5/00; A61B 5/00

(52) U.S. Cl. CPC A61B 5/01; G01J 2005/0077; A61B 5/7275; G01J 5/00

(57) ABSTRACT

A portable or semi-portable kiosk configured as a human body temperature scanning device, connected to a network that is also linked to a health history database and configured to allow or deny access to a building to a user.

This application claims priority to Provisional U.S. Patent Application Ser. No. 63/026,235, filed May 18, 2020, which is incorporated herein as if fully set forth.

### BACKGROUND

### Field of the Invention

A portable or semi-portable kiosk configured as a human body temperature scanning device, connected to a network that is also linked to a health history database and configured to allow or deny access to a building to a user.

It has been long established that certain physiological conditions are indicative of the present state of infection. Anyone routinely getting a wellness physical invariably has certain initial diagnostics taken, for example, weight, height, blood pressure, heart rate, temperature and may also submit to certain blood and urine samples for testing. Base lines are established for each individual and variance from those established base lines can indicate a current anomaly for diagnosis. Easily the least invasive and most indicative of a current infection (either bacterial or viral) is an elevated body temperature (also indicative of inflammation).

Each individual's medical history and base line figures are private and protected health information for that individual, whose permission is lawfully required for any disclosure or use of that information. Nevertheless, regarding body temperature, there are generally accepted established parameters within tolerances that are accepted as normal, and anything statistically significantly raised above that range is considered indicative of an infection state. It is however, desirable to be more accurate with an actual baseline to current comparison based on an individualized normal.

There have been many recent adaptations for automating health assessment to address rising costs of healthcare, make healthcare more accessible to anyone and make it more convenient at any time of day or night. For example, in U.S. Pat. No. 9,743,844, entitled, COMMUNITY BASED MANAGED HEALTH KIOSK AND PRESCRIPTION DISPENSEMENT SYSTEM, to Computerized Screening, Inc., disclosed is, “systems and methods that provide community based managed health kiosks and prescription drug dispensing systems, and more particularly to facilitating automated drug dispensement by a kiosk system following authorization by a remotely located health care professional who can monitor and communicate with a patient via the kiosk system.”

In another example, US Patent Application Publication No. US 2014/0330579, entitled, MEDICAL KIOSK AND METHOD OF USE, to Healthspot, Inc., disclosed is, “a medical kiosk designed to provide tele-medicine services, check-in services, and/or prescription services for a user. The medical kiosk can include a user video conferencing system that is designed to enable the user to have a real-time or near real-time tele-conference with a medical provider located remotely from the medical kiosk.”

It is unclear just how much these kiosks are in actual use and/or whether they have overcome the privacy issues. However, there also exists certain automated procedures for providing certain diagnostic tools. For example, in U.S. Pat. No. 7,020,508 entitled, APPARATUS FOR DETECTING HUMAN PHYSIOLOGICAL AND CONTEXTUAL INFORMATION, to BodyMedia, Inc., disclosed is, “a detecting apparatus includes a housing support section(s), a housing removably attached thereto, one or more sensors and a processor. An alternate apparatus measures heat flux and includes a known resistivity base member, a processing unit and two temperature measuring devices, one in thermal communication with the body through a thermal energy communicator and the other in thermal communication with the ambient environment. A further alternate apparatus includes a housing or flexible section having an adhesive material on a surface thereof for removably attaching the apparatus to the body. A further alternate apparatus includes a housing having an inner surface having a concave shape in a first direction and convex shape in a second direction substantially perpendicular thereto. Also, an apparatus for detecting heart related parameters includes one or more filtering sensors for generating filtering signals related to the non-heart related motion of the body.”

In another example, in U.S. Pat. No. 8,708,904 entitled, DEVICE UTILIZING DATA OF A USER'S CONTEXT OR ACTIVITY TO DETERMINE THE USER'S CALORIC CONSUMPTION OR EXPENDITURE, to BodyMedia, Inc., disclosed is, “a monitoring apparatus that includes a sensor device and an I/O device in communication with the sensor device that generates derived data using the data from the sensor device. The derived data cannot be directly detected by the associated sensors. Alternatively, an apparatus that includes a wearable sensor device and an I/O device in communication with the sensor device that includes means for displaying information and a dial for entering information. Alternatively, an apparatus for tracking caloric consumption and caloric expenditure data that includes a sensor device and an I/O device in communication with the sensor device. The sensor device includes a processor programmed to generate data relating to caloric expenditure from sensor data. Alternatively, an apparatus for tracking caloric information for an individual that utilizes a plurality of classification identifiers for classifying meals consumed by the individual, each of the classification identifiers having a corresponding caloric amount.

In another example, in U.S. Pat. No. 8,918,162, entitled SYSTEM AND METHOD FOR USING THREE DIMENSIONAL INFRARED IMAGING TO PROVIDE PSYCHOLOGICAL PROFILES OF INDIVIDUALS, to Francine J. Prokoski, disclosed is, “a sequence of three-dimensional thermal infrared images of the face or other area of the body is analyzed to extract time-varying maps which link skin surface temperature details to the locations of anatomical structures. Frame-to-frame quantitative variations in temperature and position of anatomical nodes of the body maps are determined for the sequence and compared to reference profiles to produce estimates of physiological condition and psychological state. The magnitude, latency, and duration of such variations at particular anatomical locations resulting from selected controlled stimuli have been determined to be strongly correlated with particular psychological states.”

Also in the prior art is the ability to actually communicate through a kiosk with a live attendant. For example, in US Patent Application Publication No. US 2013/0266925, entitled, EMBEDDED CONVERSATIONAL AGENT-BASED KIOSK FOR AUTOMATED INTERVIEWING, to Jay F. Nunamaker, JR. et al, disclosed is, “methods and systems for interviewing human subjects are disclosed. A user interface of a computing device can direct a question to a human subject. The computing device can receive a response from the human subject related to the question. The response can be received using one or more sensors associated with the computing device. The computing device can generate a classification of the response. The computing device can determine a next question based on a script tree and the classification. The computing device can direct the next question to the human subject using the user interface of the computing device.”

Finally, the ability to read temperature non-invasively is known. For example, in U.S. Pat. No. 8,663,106, entitled, NON-INVASIVE TEMPERATURE MONITORING DEVICE to BodyMedia, Inc., disclosed is, “a monitoring system comprises a module having at least one sensor and preferably skin and ambient temperature sensors within a housing. The device may be durable or disposable. The housing may be provided with certain surface features and shapes to facilitate mounting on and interface with the skin of the wearer for more accurate temperature measurement. A receiver may be provided to obtain and display data from the module. The module may also display the output data. The output data comprises both detected and derived data relating to physiological and contextual parameters of the wearer and may be transmitted directly to a local recipient or remotely over a communications network. The system is capable of deriving and predicting the occurrence of a number of physiological and conditional states and events and reporting the same as output data.”

In non-patent references, there exist kiosks with temperature screening ability. For example, there is the Easy Badges Temperature Screening Kiosk (information available at www.easybadges.com/product/temperature-sensing-kiosk-by-easy-badges/as of May 13, 2020) that is configured to be “plug and play. No tools or internet connections are required . . . [and optionally] includes more advanced features such as facial recognition, email notifications, reports, and remote monitoring.” This device is a forehead measuring device where the subject only needs to be within 0.5 to 1 meter from the unit and produces alleged accuracy to within ±0.5° C.

In another example, Protective Technologies International produces a Zortemp 1000 Body Temperature Infrared Reader (information available at www.pti-world.com/product/zortemp-1000/as of may 13, 2020) that is configured with a “touch screen” and dispenser for sanitizer, and utilizes a wrist sensor, is “designed for operating indoors and in a temperature controlled environment.” If used as stated, it has an alleged accuracy of ±0.3° C.

None of the foregoing references, alone or in combination, teach the salient and proprietary features or construction of the present disclosure, and as such, fail to be useful as an automated kiosk for determining the likely infectious state of an individual at a given moment in time via first positively identifying the individual, accessing the individual's medical records, and taking and comparing a current, accurate, non-contact temperature reading of that individual to a base line of that same individual while protecting privacy and using that information as a basis to deny or allow access through a barrier.

The present disclosure teaches several embodiments of a kiosk that have one or more means for positive identification, access to a network sufficient to allow access to an expected user's medical records with proper permissions, a means for accurately taking the temperature of the individual, and a means for opening or securing a barrier to allow or deny access of the individual through said barrier, all without any need for the individual to come into any contact with the kiosk and all automated and performed within a matter of seconds for quick, efficient, and accurate safety monitoring at a checkpoint. In a given situation, as will be detailed below, some of the described features may or may not be necessary, or others may be incorporated to address specific circumstances. In some embodiments, records may be stored and/or updated to an individual's electronic health record.

### SUMMARY

The present disclosure teaches embodiments of a kiosk comprising one or more of the following elements:
a front surface area comprising a screen, a camera, a microphone, a speaker, an identification means, and a slot area;
the slot area further comprising an at least one laser configured to have a user place their wrist in a specific area within said slot area wherein an at least one temperature sensor is configured to take a reading of said user's wrist when placed in said specific area; and
an internal processor and software configured to access a network, access all of the foregoing components, identify an individual, confirm said individual's identity and permissions, access said individual's medical records, take said individual's wrist temperature; compare said wrist temperature with said individual's medical records, analyze taking into account the current environmental conditions, determine the likelihood of present infectious state of said individual, and access a perimeter barrier mechanism to allow or deny access through said barrier;
all of the foregoing performed in an automated fashion and without the need for the individual to make contact with any component; and
update said individual's electronic health record with the current data taken.

In one embodiment, the apparatus disclosed herein facilitates the efficient, safe, automated admission of a person into a building, an airplane, a venue, or simply an area. In uncertain times when infectious diseases are rampant, it is important to have a secure method to admit healthy persons and deny admittance of sick persons to a particular area where persons will congregate to prevent spread of sickness. It is well known that when a person is sick, a person's own natural immune response produces a fever and that person's body temperature will be elevated from its normal. In one embodiment, the apparatus disclosed can perceive whether the individual currently using it is wearing a mask, or not, and can direct a person to use a mask before entry will be allowed, even if the individual is asymptomatic.

Typically, a person's normal body temperature has been set at 98.6° F. More recently, this standard has been somewhat lowered to 98.2° F. A typical adult body temperature normal can be anywhere from 97° F. to 99° F.; whereas, babies and children may have a slightly higher baseline, typically from 97.9° F. to 100.4° F. Moreover, a body temperature is not the same even throughout one day. Certain factors may influence body temperature in any given moment, such as, recent activity, time of day, age, sex, recent meal, current weather conditions, etc. Thus, unless a temperature reading is stastically and accurately excessive (generally considered to be over 102° F., but may be slightly different within specific individuals), it is less likely to determine infectious status without a baseline reading for that individual and factoring in influential factors.

The first step, though, is to have an accurate reading. While a rectal reading is commonly attributed as the most accurate reading, it is also very invasive and a generally inconvenient method. For purposes here, a reading that does not involve body contact is most desirable since that method will not be capable of passing or spreading infection, which would defeat the purpose of the testing provided herein. Infrared thermometers have become quite accurate. Some are meant to be utilized pointing toward the forehead, while others point into the ear, and still others are used with the wrist. The most accurate, least invasive, and easiest to use are those that use a wrist reading. The Uniview OET-213H-BTS1 that incorporates the Digital Detection Module provides an infrared wrist temperature sensor that if used properly, can achieve a measurement accuracy to within 0.1° C. and a deviation of less than 0.3° C. The measurement distance of the wrist to the sensor is 1 cm-3 cm, wrist exposed and angled perpendicular to the sensor. The relative environment can effect readings. Information on this unit is available at www.uniview.com/Products/Heat-Tracker/OET-213H-BTS1/available on May 13, 2020.

Thus, while other units are out there that provide facial recognition, wrist or other non-contact sensing of temperature, and other internet connected features, none are designed to optimize readings and accuracy by providing laser positioning (which also prevents unintended touching of the wrist to the sensor), a protected environment to obtain the reading (i.e., an area shielded from excessive drafts or wind), an ability to compare such reading to an actual baseline of a particular individual, identification of the individual being scanned, engagement of a current electronic health record with HIPAA consent and release, communication with a remote attendant, recognition if personal protective equipment is being used, and an automated entry through a barrier. There also is no current solution that updates and adds to an individual's electronic health record or is provided in combination with a personally accessible mobile device application to integrate and facilitate permissions, record keeping, and accessibility.

### BRIEF DESCRIPTION OF THE DRAWINGS

FIG. 1 depicts one embodiment of the present disclosure wherein the front upper functional part of a kiosk (1) is presented wherein on the left side there is a screen (2) comprising certain features which could be an infrared light camera (3), a visible light camera (4), an at least one visible light supplement lamp (5) (two are present in this iteration), an infrared light supplement lamp (6), a microphone (8), a ‘pass-through’ indicator (9), a card reading area (which could be for magnetic strip security cards, RFID cards and/or QR Code cards and the like) (10), a speaker (11), a reset button (12), and a USB interface port (13), and on the right side is an extension arm (for housing part of the converging laser system described below) creating a space between the functional left side and the right side termed here as the slot area (7).

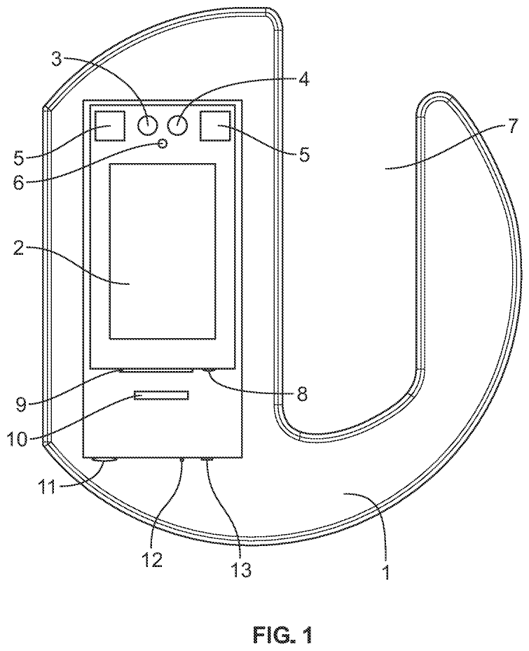

FIG. 2 depicts one embodiment of the present disclosure wherein the backside of the kiosk is presented wherein it comprises a network interface (20), a power input (21), a computer interface connector (22) and tamper proof button (23).

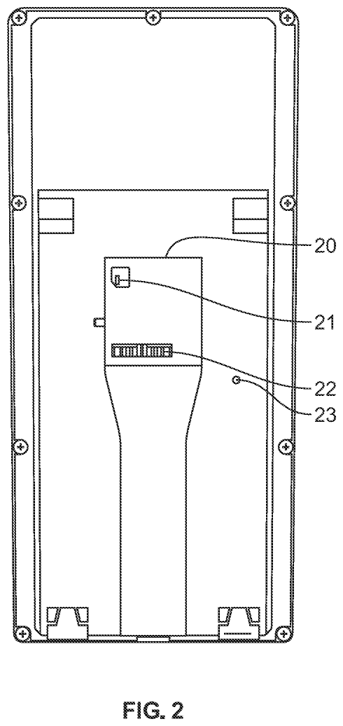

FIG. 3 depicts one embodiment of the present disclosure wherein the upper front facing functional part of a kiosk (1) is presented wherein on the left side there is a screen (2) comprising certain features as shown in FIG. 1, with an added facial recognition port (19) and shown here supported by a telescoping pedestal base.

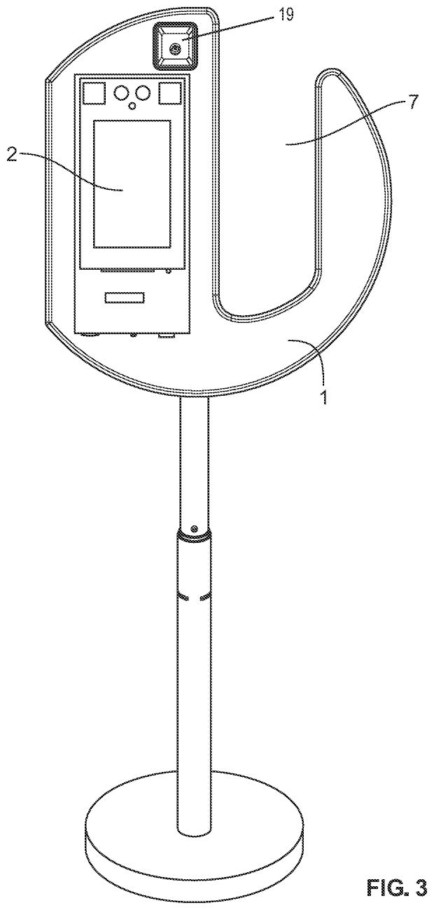

FIG. 4 depicts one embodiment of the present disclosure wherein the upper functional part of a kiosk (1) and the slot area (7) is further depicted showing where a preferred placement of the wrist of a user would go (14) and using laser lines (15) and angles to assist the user in positioning the wrist in that location.

FIG. 5 depicts one embodiment of the present disclosure in a perspective view wherein the upper functional part of a kiosk (1) and the slot area (7) is further depicted showing a preferred placement of a left side laser (16) to assist in wrist placement at a position relative to an infrared wrist temperature sensor (17).

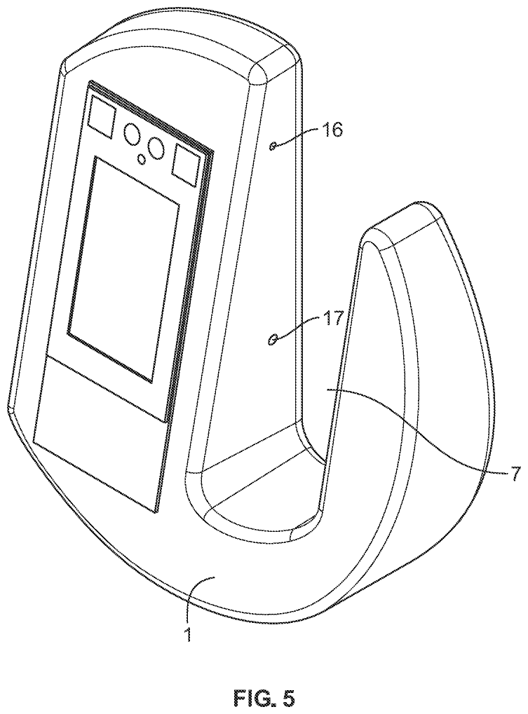

FIG. 6 depicts one embodiment of the present disclosure in an alternate perspective view wherein the upper functional part of a kiosk (1) and the slot area (7) is further depicted showing a preferred placement of a right side laser (18) to assist in wrist placement at a position relative to a temperature sensor (not seen as it is hidden in this view—see FIG. 5).

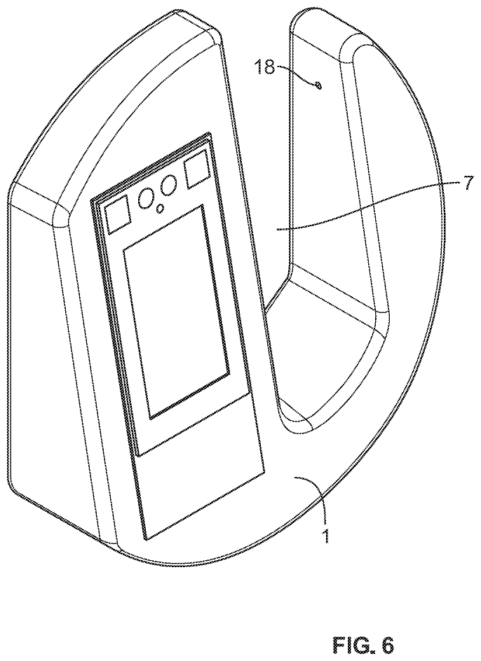

FIG. 7 depicts one embodiment of the present disclosure in a perspective view wherein the upper functional part of a kiosk (1) and the slot area (7) is further depicted showing a preferred placement of a left side laser (16) to assist in wrist placement at a position relative to an infrared wrist temperature sensor (17).

FIG. 8 depicts one embodiment of the present disclosure in an alternate perspective view wherein the upper functional part of a kiosk (1) and the slot area (7) is further depicted showing a preferred placement of a right side laser (18) to assist in wrist placement at a position relative to a temperature sensor (not seen as it is hidden in this view—see FIG. 7).

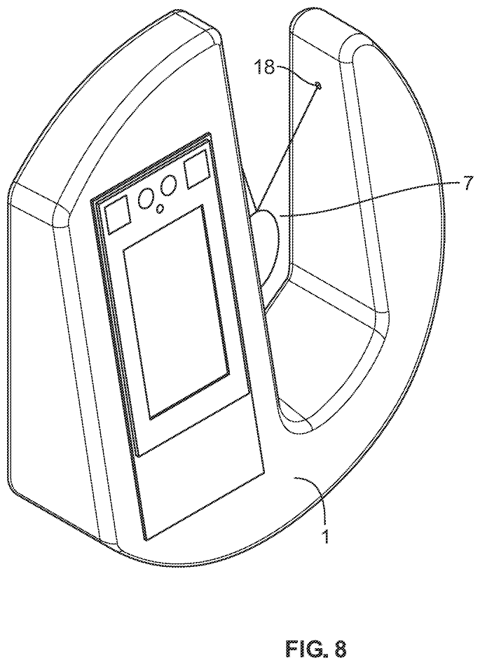

FIG. 9 depicts one embodiment of the present disclosure wherein the inside surface area of the left side of the front functional part of the kiosk forming the slot area comprising a laser (16) and a wrist temperature sensor (17) is depicted in a preferred embodiment and illustrating relative placements.

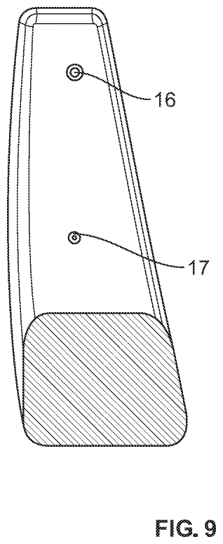

FIG. 10 depicts one embodiment of the present disclosure in an alternate perspective, almost side view wherein the upper functional part of a kiosk (1) and the slot area (7) is depicted showing a preferred placement of a left side laser (16) to assist in wrist placement at a position relative to a temperature sensor (not seen), the screen (2) and the general orientation and angles as further detailed below.

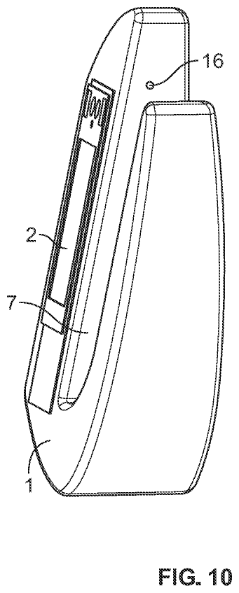

FIG. 11 depicts one embodiment of the present disclosure wherein the inside surface area of the left side of the front functional part of the kiosk forming the slot area comprising a left side laser (16) and a wrist temperature sensor (17) is depicted in a preferred embodiment and illustrating general relative placement and angles relative to the front surface screen (2) and as more detailed with specific dimensions below.

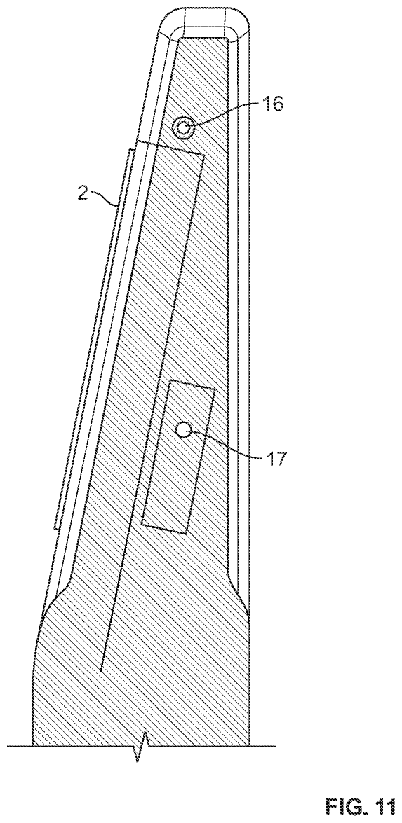

FIG. 12 depicts one embodiment of the present disclosure wherein the functionality of the kiosk as described in embodiments herein is facilitated through a mobile device application that would belong to a user and be personal to a user.

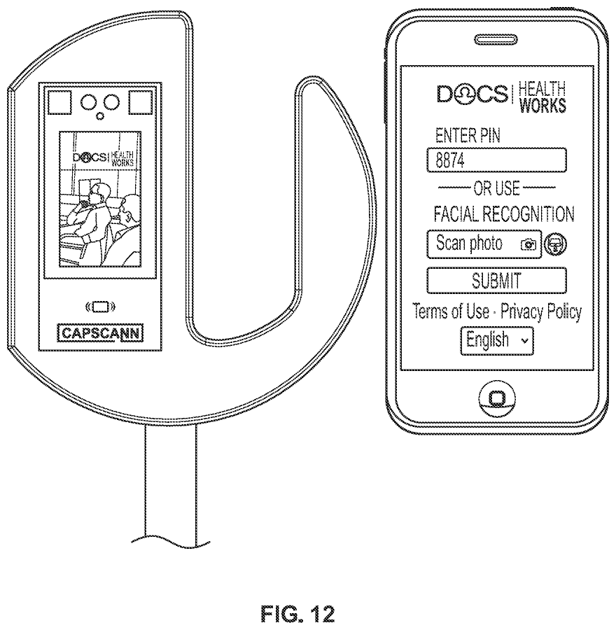

### DETAILED DESCRIPTION

For clarity of disclosure, and not by way of limitation, the detailed description of the invention is divided into the following subsections that describe or illustrate certain features, embodiments or applications of the present invention.

### Definitions

“kiosk” as used herein means any support mechanism comprising a housing to contain the various other components as described. It can be a freestanding taller support, standing on a floor, or a shorter support, standing on a desk, counter or other surface, and is generally portable. However, some kiosks may be bolted in place for security or built into other structures such as a wall to provide a permanent or semi-permanent station.

“wrist temperature sensor” as used herein means any device which can determine a body temperature in a non-contact manner when in close proximity to an area of skin designed to take a reading. In a preferred embodiment, this would refer to an infrared wrist temperature sensor device.

“laser” as used herein means any consistent, non-dangerous, streamlined light ray that can be utilized to determine and illustrate a preferred position for an object in a certain area of space.

“converging laser system” as used herein means any plurality of lasers used to create a specific area in space within which to place an object (such as a wrist) to ensure correct placement for optimal functionality of other components.

“network” as used herein means any open or closed association of computer devices connected via some mechanism, such as the Internet, an Extranet, Ethernet, a server connected to peripheral devices, and the like, and communication may be via any mechanism, such as wired connection, WiFi, Bluetooth®, and the like.

### The System and Method of the Present Invention

An apparatus as described herein may comprise one solid contiguous device with no extraneous or moving parts to jiggle, wobble, get lost, or entangled in material with the various described components set into a framework. The apparatus is designed to be used in a static manner, as a freestanding kiosk, or as a desktop model, or could also be a permanent, or semi-permanent built-in model. In an alternative embodiment, the apparatus and components as described herein could be modular and capable of connecting and disconnecting as desired.

In one embodiment, the kiosk as described herein provides an automated, non-contact, wrist scanning system capable of determining a body temperature from such wrist scan. In one embodiment, the wrist scanning system works optimally wherein the wrist is in a particular position relative to the wrist scanning system that, but for the teachings herein, would be difficult to achieve consistently or without sometimes also bumping and contacting the device itself to the user's skin. In one embodiment, to achieve the particular best position (the exposed surface of the wrist centered perpendicular to and 1 cm-3 cm away from the sensor eye), a harmless, converging laser system is concurrently used to identify the exact location in space where this area exists. In one embodiment, audible instructions can be provided to a user in order to alert the user of how to place their wrist in the slot area based on the laser positioning system.

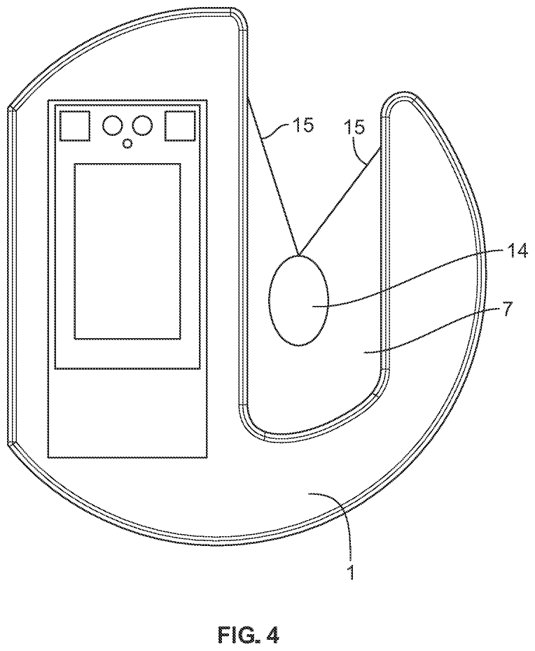

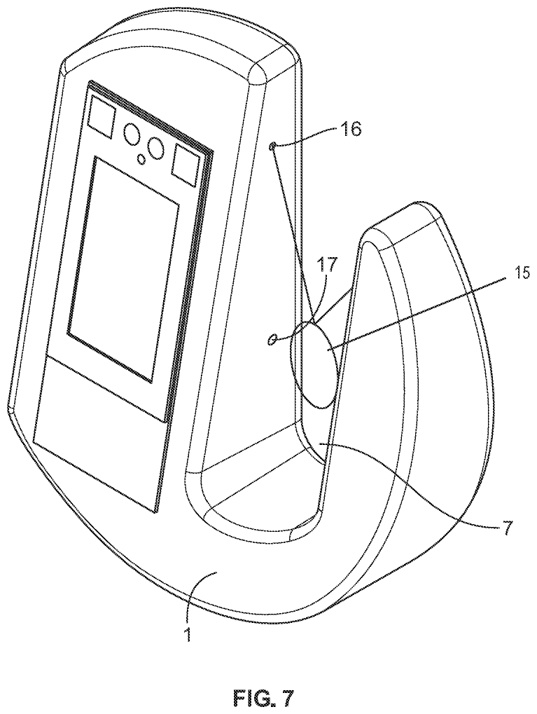

In one embodiment, in order to achieve the converging laser system, there must be a laser from at least two points, one point on either side of the wrist position area. In order to achieve this, in one embodiment here, provided is a slot area (7) as shown in the Figures, notably FIG. 4. In one embodiment, referring to FIG. 4, one left side laser (16) is configured to project a portion of a laser dot or circle (15) from the left inner surface of the slot area, a second right side laser (18) is configured to project a corresponding portion of a laser dot or circle (15) crossing from the right inner surface of the slot area, and the two light lines of the cross converge at a point (and cross) 1.5 inches above, and centered perpendicularly from, an imaginary perpendicular line emanating from the infrared wrist temperature sensor eye (17), and 1.5 cm from the left inner surface of the slot area to create a wrist placement area (14). Referring to FIG. 7, an imaginary dot placed at the center of the wrist placement area (15), would be approximately 1.5 cm below where the laser convergence point is and perpendicularly in line with the infrared wrist sensory eye (17). To achieve this, in this embodiment, the left side laser (16) emanates from a point that is approximately centered, front to back, of the inner side of the left portion of the unit forming the slot area, and approximately 3.372 in down from the top of the unit and 5.00 in above the infrared wrist sensor eye (17), and angled to project approximately 17.88° from the side wall. The right side laser (18) emanates from a point that is approximately centered, front to back, of the inner side of the right portion of the unit forming the slot area, and approximately 2.00 in down from the top of the unit and angled to project approximately 37.10° from the side wall. In one embodiment, the converging laser system utilizes a cross pattern and a dot or circle and when the dot or circle is in the center of the cross, then the alignment is correct and one line in the cross orients the hand, wrist, and arm perpendicular to the wrist sensor eye and the crossing line in the cross orients in the horizontal location of the writs sensor eye. The combination of the circle or dot provides the third dimension in space. In one embodiment, the slot area is approximately 4.5 in wide, the left side of the system comprising the screen, is approximately 8 in wide and the right side is approximately 3 in wide at its widest part. In one embodiment, the unit is approximately 5 in thick, front to back at its lower portion and tapering thinner as it ascends toward the top of the unit to approximately 2 in thick, front to back at the top portion, with the front surface, including the screen, angled from front to back. In one embodiment, from the lower portion of the slot area, to the top of the left side of the unit is approximately 15 in.

In one embodiment, the kiosk will comprise a screen (2), which can be utilized to provide the user instructions on use and report to the user certain results; a camera (4), which can be utilized in conjunction with facial recognition software to identify and store the identity of a user (or have a dedicated facial recognition port (19)); a speaker (11), which can be utilized to provide audible instructions, and also to communicate live with a user; a microphone (8), which can be utilized by a user to interact with automated instructions and/or used to communicate live with an administrator; and an additional feature such as a card reader (10), a device that can scan a QR code, a magnetic security card, an RFID card, and the like, to further identify a user and unlock other pre-enabled features and/or identify medical records, provide permissions, start a process, and the like.

In one embodiment, the kiosk, through a camera component and facial recognition software, will be able to recognize pre-enabled users upon such user walking up to the kiosk, and store additional facial features as newly learned. In this manner, the kiosk can save time as to expected entrants at a threshold.

In one embodiment, software will be configured to enable the automatic lifting of an entryway gate, undo a door lock, and the like, to provide entrance to a visitor at the threshold upon the finding of a non-elevated temperature, and in the alternative, upon the finding of an elevated temperature, lock a door, secure a gate, and record the identity of the elevated temperature person and make a report to building security or human resources.

In one embodiment, the software and connectivity of the kiosk will, with permissions and positive identification, access medical records of a user to create a more accurate baseline for that individual and the software will be configured to adjust the threshold temperature for admittance or denial of admittance based on the surrounding temperature, the individual's baseline temperature, the individual's most recent temperatures, the time of day, and other known factors affecting body temperature. In one embodiment, the kiosk may also add to the medical records of the individual based on the reading at that moment.

In one embodiment, when an abnormally elevated or abnormally depressed temperature is recorded, instructions may be given for a second test in five minutes or some other pre-determined or requested time interval to allow for the individual to adjust to the surrounding environment or otherwise normalize. In some embodiments, suggestions will be given to the user to ensure that the user is performing the readings correctly by exposing the wrist and properly placing it according to the converging laser system.

In one embodiment, the kiosk may flag individuals for additional testing and obtain the results of such testing when completed.

In one embodiment, the kiosk may be pedestal mounted, or counter top mounted, or turnstile mounted, or permanently mounted akin to an ATM at a bank.

In one embodiment, a voice intercom system can be integrated to communicate live with building security, human resources, a doctor's office, a building resident, or any combination of desired contacts to facilitate entry into the area and monitoring of activity.

In one embodiment, the kiosk may be integrated with voice prompting for usage.

In one embodiment, the screen could be utilized for advertising revenue.

In one embodiment, the slot area, in addition to providing a converging laser system for accurate placement of the wrist in position for reading by the wrist temperature sensor, the slot area also protects against drafts, wind, and other environmental factors that could affect temperature readings. In this manner, instead of the claimed 0.54° F. accuracy of competitors, the accuracy here can be achieved at 0.18° F. This, when combined with a more accurate baseline specific to an individual, creates the optimal environment to detect temperature abnormalities.

In one embodiment, the slot area is replaced with a substantially circular hole to put a wrist through, which would create even more environmental protection, but make seeing the laser lines more difficult. In this embodiment, the initial hole could be relatively large, but then when the wrist has stopped moving within the area, the kiosk can then activate and the converging laser system can be tied to a moveable wrist sensor mechanism and instead of the wrist moving into position relative to the wrist sensor, the wrist sensor could move into position relative to the wrist, creating even more accurate and protected readings.

In one embodiment, either in conjunction with or as a standalone replacement, a tear duct temperature measurement functionality will exist. Research shows that one of the most reliable and accurate methods to detect elevated body temperature is through use of the eye canthus (a small area over the tear duct). Skin facial temperature and its correlation to core body temperature as measured through this tear duct area has shown to be most reliable and accurate. However, the thermal imaging cameras used for such temperature detection require certain parameters be met. The camera must project a sufficient number of pixels over the eye canthus to have an adequate spatial resolution.

In one embodiment, the facial recognition software and camera system will isolate the eye canthus to project sufficient pixels over the eye canthus to create an adequate spatial resolution for proper readings while the user uses the wrist temperature functionality and based on all readings, a proprietary algorithm can provide even greater accuracy of current core body temperature than any reading alone.

In one embodiment, there could be a mobile device application capable of integration with the unit such that any functionality of the unit desirable to be controlled by an individual user may be controlled and could provide for permissions of the gathering, use and recording of medical information, providing codes for access for a pre-determined period of time without the need for further testing, and updating security protocols. The associated app could have a feature for login to provide a layer of security, or facial recognition of both the unit and the app on a mobile device could trigger a login event for ease of access.

### EXAMPLES

The present invention is further illustrated, but not limited by, the following examples.

A pandemic has occurred and buildings are on lockdown. In order to allow persons back into buildings, it is a requirement that individuals be tested for present illness. The least invasive, most efficient, and reliable manner to know if an individual is presently infected with a pathogen, is to read for an elevated body temperature. By using the kiosk as described herein, multiple individuals can be quickly, accurately, and efficiently, with no potential of spread of pathogen through touching, test and admit persons into a building on lockdown, and keep out individuals who test with elevated temperature, and report them for further testing consistent with protocol at a given time, and all while complying with privacy laws.

In one embodiment, the kiosk, or more than one kiosk, can be utilized, linked to common records. Since the kiosks can scan temperatures in approximately 0.2 seconds, and read faces and/or QR codes or other identifying indicia and open records also, within seconds, the only limiting factor for speed is really a user getting his or her wrist into position to be scanned. Once scanned, if approved, the gate threshold can be opened and the individual admitted, all within 5 to 30 seconds. Thus, each kiosk can admit up to seven hundred persons per hour, all automated, and more accurately than anything known today based on the described improvements.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

Each and every feature described herein, and each and every combination of two or more of such features, is included within the scope of the present invention provided that the features included in such a combination are not mutually exclusive.

What is claimed is:

1. A kiosk comprising:
a front surface area comprising a screen, a camera, a microphone, a speaker, an identification means, and a slot area;
the slot area further comprising an inner left surface area comprising an at least one laser and a temperature sensor, an inner right surface area comprising an at least one laser, said at least one laser on said left inner surface configured to generate a laser circle and said at least one laser on said right inner surface configured to generate a laser cross wherein the laser cross lines converge at a point approximately one and one-half inches above where an imaginary perpendicular line emanating from said temperature sensor would be and 1.5 cm from the face of said sensor; and
an internal processor and software configured to access a network, access said screen, said camera, said microphone, said speaker, said identification means, said lasers, said temperature sensor, and configured to initially identify an individual, confirm said individual's identity and permissions, access said individual's medical records, take said individual's wrist temperature upon said individual placing his exposed wrist at the convergence of the laser lights; analyze taking into account for current environmental conditions, compare said wrist temperature with said individual's medical records, determine the likelihood of present infectious state of said individual, and allow or deny access through a perimeter barrier mechanism;
all of the foregoing performed in an automated fashion and without the need for the individual to make contact with any component.

2. The kiosk of claim 1, wherein said slot area is replaced with a hole.

3. The kiosk of claim 1, wherein, said kiosk is mounted on a pedestal.

4. The kiosk of claim 1 wherein it is configured to be built-in to an existing wall near an entryway.

5. The kiosk of claim 1 wherein said internal processor and software is further configured to update a user's electronic data with the current data gathered.

6. The kiosk of claim 1 wherein it further comprises an eye canthus thermal imaging camera and facial recognition isolation of the eye canthus for a proper eye canthus reading.
